(19) United States
(12) Patent Application Publication
(10) Pub. No.: US 2018/0360855 A1
(43) Pub. Date: Dec. 20, 2018

(54) THE FORMULATION COMPRISING PHOSPHOMYCINE AND ASCORBIC ACID FOR THE TREATMENT OF BACTERIAL URINARY SYSTEM INFECTIONS

(71) Applicant: Yusuf Toktamis ÖGÜN, istanbul (TR)

(72) Inventors: Yusuf Toktamis ÖGÜN, istanbul (TR); Mehmet AGCAYAZI, istanbul (TR); Hatice CANBAGI, istanbul (TR)

(73) Assignee: Yusuf Toktamis ÖGÜN, istanbul (TR)

(21) Appl. No.: 16/060,961

(22) PCT Filed: Dec. 25, 2015
(86) PCT No.: PCT/TR2015/050271

§ 371 Date: Jun. 11, 2018

(51) Int. Cl. A61K 31/665; A61K 31/375; A61K 9/00; A61P 31/04

(52) U.S. Cl. CPC A61K 31/665; A61P 31/04; A61K 9/009; A61K 31/375

(57) ABSTRACT

The present invention is related to a pharmaceutical formulation in the form of a sachet which comprises ascorbic acid and phosphomycine trometamol, to be used in treating urinary system infections caused by pathogens sensitive to phosphomycine. The formulation is characterized in that phosphomycine is enabled to be more efficient in treating urinary system infections depending on the fact that ascorbic acid reduces the pH of urine.

### CROSS REFERENCE TO THE RELATED APPLICATIONS

This application is the national phase entry of International Application No. PCT/TR2015/050271, filed on Dec. 25, 2015, the entire contents of which are incorporated herein by reference.

### TECHNICAL FIELD

The present invention is related to the preparation of a combination formulation to treat the urinary system, comprising the phosphomycine active ingredient having synergic effects together with ascorbic acid. Said product is characterized in that it is in sachet form.

### BACKGROUND

Phosphomycine trometamol is a synthetic bactericidal antibiotic. It shows effect by inhibiting the first stage of the peptidoglycan synthesis of bacteria on the cell wall. It has been described primarily, in the patent numbered U.S. Pat. No. 3,914,231 that it was effective both on gram negative and gram positive microorganisms, and that it was a molecule that was obtained from streptomyches species. Some of the microorganisms susceptible to Phosphomycine are species such as E. coli, Citrobacter species, Klebsiella species, Proteus species, Staphylococcus species, Salmonella, S. faecalis, P. aeruginosa and Serratia species.

Phosphomycine is available in the market in 3 gram sachet and effervescent tablet forms. The other molecule present inside the combination which is ascorbic acid is a vitamin soluble in water and it is not stored in the body. The vitamin C that is required is taken together with diet. It also has antioxidant characteristics.

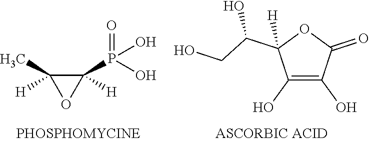

U.S. Pat. No. 7,303,755 (Zambon) describes a pharmaceutical composition comprising Phosphomycine Trometamol in which arginin and lysine and some citrate, phosphate, carbonate, tartrate salts are used.

A pharmaceutical formulation comprising glycine and at least an excipient together with Phosphomycine Trometamol has been described in the European Patent Application (Arafarma) Numbered EP2656846. It is defined that the excipients is not from the group of sugar or sugar alcohols.

The Turkish application numbered TR2012/05003 (Sima) is related to an effervescent type pharmaceutical product comprising Phosphomycine Trometamol.

ES495870 numbered Spanish Application describes a sachet formulation comprising phosphomycine trometamol, sodium carboxymethylcellulose, lactose, titanium dioxide, orange aroma and sugar.

ES2020790 numbered Spanish application (Zambon) is related to a formulation comprising phosphomycine trometamol, saccharine, aroma and sugar.

The Spanish application numbered ES2244333 (Simbec Ibérica) is related to a pharmaceutical formulation comprising phosphomycine trometamol and the excipient system comprising mannitol and/or xylitol and an artificial sweetening agent which is selected from the group of acesulfame, aspartame, saccharin, alitame and/or cyclamate.

### SUMMARY

It has been noted in, in vitro studies that Phosphomycine showed efficiency on susceptible bacteria in 30 minutes. Phosphomycine shows its effect by reducing the motion capabilities and synthesis of bacterial fimbriae. It prevents both gram positive and gram negative pathogens to adhere onto the urinary system epithelium, the ureters and urethra's inner surface and also prevents their colonization herein. Moreover, it can have anti adhesive efficiency even in values under the minimum efficient concentration. This effect is the most significant step of pathogenesis in the urinary system. The reason for this is that pathogens may adhere to the epithelium cells in the urinary system as they have adhesive features thereby preventing the flow of urine and they may accumulate on the surface of the bladder and enter into tissues.

It has also been noted that when phosphomycine is examined in vitro, its efficiency is affected by environmental conditions. It has been noted following the tests carried out that the efficiency of Phosphomycine is reduced in the presence of Alkali pH, glycose, phosphate, and sodium chloride.

It is known that the pH of urine changes according to the acidic or alkali agents taken externally. This information is used when some medicines that are excreted via urine, need to be excreted earlier than usual from the body (ion trap). Additionally, the efficiency of some antimicrobial agents in the urinary system may increase or decrease, depending on the pH of urine. For example, the efficiency of tetracycline, methycillin, cloxacillin, methenamine and nitrofurantoin is higher in acidic urine. Similarly, gentamicin, clindamycin, lincomycin, cephalosporines and erythromycin have higher efficiency in alkali urine. The efficiency of Chloramphenicol, Penicillin G, colistin, vancomycin and Nalidixic acid is not affected by the pH of urine.

When the urine pH needs to be changed by means of an external intervention, ammonium chloride, sodium acid phosphate or ascorbic acid is given in order to acidify the urine or sodium bicarbonate is given in order to ensure that the urine pH is alkali.

It has been noted that the pH increased depending on the increase in the amount of water during the absence of ascorbic acid and that the pH significantly decreased in the presence of ascorbic acid according to the scientific study we have carried out. This result proves that when there is a significant decrease in the pH of urine in the presence of ascorbic acid, efficacy of phosphomycine shall increase during the treatment of urinary system infections (Table 1).

TABLE 1
Comparison of Phosphomycine Trometamol and Ascorbic Acid
and the pH of solutions of Phosphomycine Trometamol
having the same concentrations

| | Phosphomycine | Phosphomycine Trometamol + |
|---|---|---|
| | Trometamol | Ascorbic acid |
| | (5.631 g) | (5.631 g + 1.0 g) |
| pH in 50 ml water | 3.608 | 2.869 |
| pH in 100 ml water | 3.743 | 2.934 |
| pH in 150 ml water | 3.824 | 2.978 |

The present invention describes a pharmaceutical formulation comprising Phosphomycine Trometamol together with ascorbic acid. Said pharmaceutical formulation is in sachet form.

The characteristic of the formulation comprising Phosphomycine Trometamol together with ascorbic acid is that ascorbic acid reduces the pH of urine, while phosphomycine then becomes more effective in treating urinary system infections.

In addition to, the formulation comprises suitable excipients in order to prepare the pharmaceutical product together Phosphomycine Trometamol and ascorbic acid.

The characteristic of sachet formulations is that it is a pharmaceutical form which is superior to other oral solid forms when evaluated in terms of the fact that it is easily soluble, and faster in dispersion when compared to tablets or capsules.

The formulation of the present invention is also in form which can be used easily and more suitably for special patient groups such as those having difficulty in swallowing or geriatric or paediatric patients.

### DETAILED DESCRIPTION

The present invention is related to a pharmaceutical formulation in sachet form comprising two active ingredients, one of them has weak acidic features, and other is used in treating urinary system infections. The active ingredients subject to the invention are Phosphomycine Trometamol which is a wide spectrum antibiotic used in generally treating urinary system infections and ascorbic acid having weak acidic features also known as Vitamin C.

The formulations of the present invention comprise at least an excipient in the required and sufficient amount together with phosphomycine trometamol and ascorbic acid active agents.

As the pharmaceutical form of the present invention is a sachet, the formulation comprises at least a sweetener and at least an aromatic ingredient as excipients.

Phosphomycine used in the formulation subject to the invention can be in the form of phosphomycine that are pharmaceutically acceptable salts, hydrates, solvates, esters, enantiomers, diastereomers or combinations thereof. The phosphomycine can be amorphous or may have a crystal structure or a mixture thereof in terms of polymorphic structure.

The phosphomycine which is to be used in the formulations of the invention is preferably in salt form. Preferably phosphomycine trometamol is used.

The ascorbic acid that is to be used in formulations of the invention is a weak acidic agent and can be found in chemically different isomeric structures, it is found in crystal, amorphous form or as a mixture thereof in terms of polymorphic structure.

The ascorbic acid used in formulations of the invention is an L isomer that is naturally available, and which has superiority in efficiency in comparison to D isomer. The solubility in water of L-ascorbic acid (L-AA) is high. It has a durable structure when it is crystal form. However, it has a tendency for oxidation in aqueous solutions and it is one of the least stable agents among vitamins.

D-Ascorbic acid which is another chemical form of L-AA, has very little biological activity whereas D-isoAscorbic Acid=eritorbic acid does not have any biological activity. However, these three forms of ascorbic acid are all used in the food sector and as nutrition supplements as they have antioxidant characteristics.

The formulations according to the invention comprises Phosphomycine Trometamol between 10% to 95%, preferably 20% to 90%, more preferably 40% to 80%, most preferably 60% to 65% in weight according to total sachet weight.

The formulations according to the invention comprises Ascorbic acid between 2% to 40%, preferably 5% to 30%, more preferably 5% to 25% in weight according to total sachet weight.

The phosphomycine base: ascorbic acid ratio in the pharmaceutical formulation is between 1:1 to 10:1 in weight, preferably 1:1 to 10:1 in weight. A sachet as unit dosage form comprises approximately 5,653 g Phosphomycine Trometamol equivalent 3.0 gram of Phosphomycine base and approximately 0.025 to 3.0 grams, preferably 0.5 grams to 2.0 grams of L-Ascorbic Acid.

A sachet as unit dosage form comprises approximately 5,653 g Phosphomycine Trometamol equivalent 3.0 gram of Phosphomycine base and approximately 0.025, 0.3, 0.5, 0.75, 1.0, 1.25, 1.5, 1.75, 2.0, 2.25, 2.50, 2.75 or 3.0 grams of L-Ascorbic Acid. Sachet formulations of the invention is powder or granule form or a mixture thereof.

Pharmaceutical products can comprise aspartame, acesulfame, potassium, dextrose, fructose, saccharine, maltitol, mannitol, sorbitol, cyclamate, sucralose, sucrose, or different crystal forms or solutions thereof as sweeteners. The sweetener to be used in the formulation of the invention can be any one or more of the above or a mixture thereof.

Various aromas can be used to provide taste or scent or to mask bad taste or scents in pharmaceutical products. Examples of these can be strawberries, orange, mint, menthol, cherries, lemon, banana, aniseed, vanilla, chocolate or mixtures thereof. Aromas can be naturally extracted from plants but they can also be produced synthetically. The aroma to be used in the formulation of the invention can be one or more of the examples given above or it can be a mixture thereof.

The formulation of the invention comprises sucrose, saccharine sodium, orange aroma and tangerine aroma as sweeteners and aroma.

As a general approach, the taste of the pharmaceutical formulations in the sachet form should not be disturbing to the patient and it should positively affect the compliance of the patient. For this reason, the types and optimum amounts of the sweetener(s) and aroma(s) to be used is organoleptically determined.

The formulations according to the invention comprises at least a sweetener between 5% to 30% by weight according to total sachet weight.

The formulations according to the invention comprises at least an aroma between 0.5% to 10% by weight according to total sachet weight.

### EXAMPLES

TABLE 1
Sachet formulations comprising phosphomycine trometamol equivalent to 3.0 g
of phosphomycine base and ascorbic acid between 0.5 to 2 g.

| | g/sachet | % | g/sachet | % | g/sachet | % |
|---|---|---|---|---|---|---|
| Phosphomycine trometamol | 5.63 | 62.57 | 5.63 | 62.57 | 5.63 | 62.57 |
| (3.0 g Phosphomycine base) | | | | | | |
| Ascorbic acid | 1.00 | 11.11 | 0.50 | 5.56 | 2.00 | 22.22 |
| Sucrose | 2.21 | 24.59 | 2.71 | 30.14 | 1.21 | 13.48 |
| Saccharine sodium | 0.02 | 0.18 | 0.02 | 0.18 | 0.02 | 0.18 |
| Orange aroma | 0.07 | 0.78 | 0.07 | 0.78 | 0.07 | 0.78 |
| Tangerine aroma | 0.07 | 0.78 | 0.07 | 0.78 | 0.07 | 0.78 |
| Total sachet weight | 9.00 | 100.00 | 9.00 | 100.00 | 9.00 | 100.00 |
| Phosphomycine base/ | 3:1 | | 6:1 | | 3:2 | |
| Ascorbic acid | | | | | | |

The example formulations comprise an equal amount of phosphomycine trometamol and variable amounts of ascorbic acid. The amount of ascorbic acid per sachet has been formulated in 3 different doses as 0.5 g, 1 g, and 2 g.

The production process involves the sieving of dry powder and mixing thereof, followed by filling said powder into the sachets.

The pharmaceutical formulation subject to the invention is used in treating urinary system infections.

1. A pharmaceutical formulation in a sachet form, comprising Phosphomycine base or pharmaceutically acceptable salts, hydrates, solvates, esters, enantiomers, diastereomers or mixtures thereof; and Ascorbic acid.

2. The A pharmaceutical formulation according to claim 1, wherein a ratio of Phosphomycine base to Ascorbic acid is 1:1 to 10:1 by weight.

3. The pharmaceutical formulation according to claim 1, wherein the pharmaceutical formulation comprises 3.0 gram phosphomycine base equivalent of phosphomycine salt.

4. The pharmaceutical formulation according to claim 1, wherein the pharmaceutical formulation comprises Phosphomycine Trometamol.

5. The pharmaceutical formulation according to claim 1, wherein the pharmaceutical formulation comprises L-Ascorbic Acid.

6. The pharmaceutical formulation according to claim 5, wherein the pharmaceutical formulation comprises 0.25 grams to 3 grams of L-Ascorbic Acid in a unit dosage form.

7. The pharmaceutical formulation according to claim 6, wherein the pharmaceutical formulation comprises 0.5 grams to 2 grams of L-Ascorbic Acid in the unit dosage form.

8. The pharmaceutical formulation according to claim 1, wherein the pharmaceutical formulation comprises Phosphomycine trometamol equivalent to 3.0 grams of Phosphomycine base and 0.5 grams of L-Ascorbic Acid in a unit dosage form.

9. The pharmaceutical formulation according to claim 1, wherein the pharmaceutical formulation comprises Phosphomycine trometamol equivalent to 3.0 grams of Phosphomycine base and 1 gram of L-Ascorbic Acid in a unit dosage form.

10. The pharmaceutical formulation according to claim 1, wherein the pharmaceutical formulation is a powder, granule or a mixture thereof.

11. A method for treating urinary system infections, the method comprising:
utilizing a pharmaceutical formulation comprising Phosphomycine base or pharmaceutically acceptable salts, hydrates, solvates, esters, enantiomers, diastereomers or mixtures thereof and Ascorbic acid;
wherein the pharmaceutical formulation is in a sachet form.
